(19) United States
(12) Patent Application Publication
(10) Pub. No.: US 2020/0345592 A1
(43) Pub. Date: Nov. 5, 2020

(54) MULTILAYER TYPE POWDER-CONTAINING COSMETIC PREPARATION

(71) Applicant: SHISEIDO COMPANY, LTD., Tokyo (JP)

(72) Inventors: Kanako NOGUCHI, KANAGAWA (JP); HIRUMA Yukiko, KANAGAWA (JP); KAWANO Sayoko, KANAGAWA (JP); FUJITA Junko, KANAGAWA (JP)

(73) Assignee: SHISEIDO COMPANY, LTD., Tokyo (JP)

(21) Appl. No.: 16/642,597

(22) PCT Filed: Aug. 29, 2018
(86) PCT No.: PCT/JP2018/031885

§ 371 Date: Feb. 27, 2020

(30) Foreign Application Priority Data
Aug. 31, 2017 (JP) 2017-167781

(51) Int. Cl. A61K 8/03; A61K 8/02; A61K 8/34; A61K 8/25; A61K 8/29; A61K 8/894; A61K 8/36; A61K 8/81; A61K 8/26

(52) U.S. Cl. CPC A61K 8/03; A61K 8/022; A61K 8/34; A61K 8/25; A61K 8/29; A61K 2800/874; A61K 8/36; A61K 8/81; A61K 8/26; A61K 2800/10; A61K 8/894

(57) ABSTRACT

The present invention provides a multilayer type powder-containing cosmetic preparation which can be variously formulated and in which the powder ingredient has excellent re-dispersibility without being required to have undergone a surface treatment. The a multilayer powder-containing cosmetic preparation includes (a) to (c) components: (a) a 60-95% by mass lower alcohol; (b) a powder ingredient (excluding a (c) component); and (c) a finely particulate powder which has an average primary-particle diameter of 10-550 nm and includes silica or titanium dioxide as a core.

### TECHNICAL FIELD

The present invention relates to a multilayer type powder-containing cosmetic preparation (multilayer powder-containing cosmetic) which is used by shaking (re-dispersing) a powder homogenously, i.e., shaken to re-disperse the powder when used. More specifically, the present invention relates to a multilayer type powder-containing cosmetic preparation (i.e., multilayer cosmetic containing powder) containing a lower alcohol such as ethanol and having excellent re-dispersibility of the powder.

### BACKGROUND ART

There is a multilayer type powder-containing cosmetic, that is a liquid formulation containing a powder and has an appearance wherein the powder component precipitates and forms multilayers when left to stand. Such a cosmetic gives not only a cooling sensation when applied but also a dry touch due to the powder component if its liquid layer contains a lower alcohol such as ethanol, and therefore, the cosmetic can provide a feeling of use suitable for deodorant cosmetics, astringent cosmetics and the like.

Such a multilayer type powder-containing cosmetic is used after the powder and the liquid are homogenized by shaking to re-disperse the powder component, so that it is essentially required that the powder component has good re-dispersibility (in liquid). Conventional techniques for improving the re-dispersibility of a powder component in a multilayer type powder-containing cosmetic include (1) addition of a substance that promotes re-dispersion (re-dispersibility improver), and (2) surface treatment of the powder.

Patent Document 1 discloses that, the re-dispersibility of the zeolite powder was improved by adding zinc para-phenolsulfonate as a re-dispersibility improver in the presence of glutathione and/or L-cysteine, in a two-layer type powder-containing cosmetic containing an antibacterial zeolite powder and containing 50 to 90% by mass of ethanol as a base material.

Patent Document 2 states that a two-layer type powder-containing cosmetic having an excellent powder re-dispersing property is obtained by subjecting the surface of the powder to a surface hydrophobizing treatment with silicone and adding a polyether-modified silicone having an HLB of 5 or less. Moreover, Patent Document 3 states that, in an aqueous multilayer type cosmetic having an aqueous layer consisting of two or more layers, the cosmetic has excellent cooling sensation and make-up durability, and does not easily change color during use, and also has good re-dispersibility of the powder, by providing the top layer with a powder dispersion layer containing a powder having a surface coated with a surface treating agent containing a silicone phosphoric acid triester having a specific structure.

However, the substance that promotes re-dispersion varies depending on the type (property of the material) of the powder component to be re-dispersed, so that the components that can be added are limited, which results in restriction of a variety of formulations. On the other hand, there are problems such that the surface treatment of a powder is time- and cost-consuming.

### CITATION LIST

### Patent Documents

JP 4850304 B

JP 2009-234994 A

JP 5881162 B

### SUMMARY OF INVENTION

### Technical Problem

Accordingly, an object of the present invention is to provide a multilayer type powder-containing cosmetic that can be prepared in a wide range of formulation and provide an excellent re-dispersibility even if none of a surface-treated powder is mandatorily used.

### Solution to Problem

The present inventors have conducted diligent studies to attain the object and consequently completed the present invention by finding that the re-dispersibility of various powder components can be improved by using a specific fine particle powder having an average primary particle diameter of 10 to 550 nm.

Specifically, the present invention provides a multilayer type powder-containing cosmetic, comprising the following components (a) to (c):

(a) 60 to 95% by mass of a lower alcohol;
(b) a powder component (excluding component (c) below); and
(c) a fine particle powder having an average primary particle diameter of 10 to 550 nm and comprising silica or titanium dioxide in a core of the fine particle powder.

### Advantageous Effects of Invention

The multilayer type powder-containing cosmetic of the present invention has excellent re-dispersibility of the powder component, and the powder component is homogenously re-dispersed simply by shaking by hand. In the multilayer type powder-containing cosmetic of the present invention, it is not necessary to perform a surface treatment on the powder component, so that the production process can be simplified and the production cost can be suppressed. Moreover, it is not necessary to add a re-dispersibility improver tailored to the powder component, so that the kind of acceptable powder components are not limited, and a variety of the formulations can be created.

### DESCRIPTION OF EMBODIMENTS

The “multilayer (type) powder-containing cosmetic” according to the present invention is being separated into a plurality of layers (at least two layers of a powder layer and a liquid layer) in a stationary state, and the liquid layer forms one layer or two or more layers. When the liquid layer forms two or more layers, layers may be clearly separated each other to form multi-layers, or each layer may be a suspension layer, in which each layer is mixed to make a suspension.

Hereinafter, the multilayer type powder-containing cosmetic of the present invention (hereinafter, also simply referred to as “powder-containing cosmetic” or “cosmetic”) will be described in detail.

The multilayer type powder-containing cosmetic of the present invention is a liquid formulation containing a powder, and the liquid component (base material) contains (a) a lower alcohol. The lower alcohol used in the present invention is preferably a lower alcohol having 1 to 6 carbon atoms, and examples thereof include ethanol, methanol, propanol, butanol, and isopropanol. Among these, it is preferable to use ethanol.

The content of the lower alcohol in the powder-containing cosmetic of the present invention is 60 to 95% by mass. For example, it can be 62% by mass or more, 65% by mass or more, or 70% by mass or more, and for example, it can be 93% by mass or less, 92% by mass or less, or 90% by mass or less. By using a lower alcohol as a base material, the lower alcohol volatilizes to give a cooling sensation when applied to the skin.

The powder component (b) included in the powder-containing cosmetic of the present invention is not particularly limited as long as it is a powder that can be included in a cosmetic or the like. However, those corresponding to the following component (c) are excluded from component (b). The shape and size of the powder component (component (b)) in the powder-containing cosmetic of the present invention are not particularly limited. In addition, those subjected to a surface treatment and those not subjected to a surface treatment are included.

Specific examples of the powder component include: inorganic powders such as extender pigments (for example, talc, kaolin, mica, sericite, muscovite, phlogopite, synthetic mica, lepidolite, biotite, vermiculite, magnesium carbonate, calcium carbonate, aluminum silicate, barium silicate, calcium silicate, magnesium silicate, strontium silicate, metal tungstate, magnesium, barium sulfate, calcined calcium sulfate (calcined gypsum), calcium phosphate, fluorapatite, hydroxyapatite, ceramic powder, metal soap (for example, zinc myristate, calcium palmitate, aluminum stearate), boron nitride, zeolite, aluminum potassium sulfate (calcined alum), silica (however, those with an average primary particle diameter of more than 0.55 μm), and the like); resin powders (for example, polyamide resin powder (nylon powder), polyethylene powder, polyalkylacrylate powders such as polymethyl methacrylate, polystyrene powder, copolymer resin powder of styrene and acrylic acid, benzoguanamine resin powder, polytetrafluoroethylene powder, cellulose powder, crosslinked silicone powder, and the like); inorganic white pigments (for example, titanium dioxide (however, those having an average primary particle diameter of more than 0.55 μm), zinc oxide, and the like); inorganic red pigments (for example, iron oxide (red iron oxide), iron titanate, and the like); inorganic brown pigments (for example, γ-iron oxide and the like); inorganic yellow pigments (for example, yellow iron oxide, yellow ocher, and the like); inorganic black pigments (for example, black iron oxide, lower titanium dioxide, and the like); inorganic purple pigments (for example, manganese violet, cobalt violet, and the like); inorganic green pigments (for example, chromium oxide, chromium hydroxide, cobalt titanate, and the like); inorganic blue pigments (for example, ultramarine, iron blue, and the like); pearl pigments (for example, titanium dioxide-coated mica, titanium dioxide-coated bismuth oxychloride, titanium dioxide-coated talc, colored titanium dioxide-coated mica, bismuth oxychloride, fish scale guanine, and the like); metal powder pigments (for example, aluminum powder, copper powder, and the like); organic pigments such as zirconium, barium or aluminum lake (for example, Red No. 201, Red No. 202, Red No. 204, Red No. 205, Red No. 220, Red No. 226, Red No. 228, Red No. 405, Orange No. 203, Orange No. 204, Yellow No. 205, Yellow No. 401, and Blue No. 404, Red No. 3, Red No. 104, Red No. 106, Red No. 227, Red No. 230, Red No. 401, Red No. 505, Orange No. 205, Yellow No. 4, Yellow No. 5, Yellow No. 202, Yellow No. 203, Green No. 3 and Blue No. 1, and the like); and natural pigments (for example, chlorophyll, β-carotene, and the like).

The content of the powder component (component (b)) in the powder-containing cosmetic of the present invention is usually 0.1 to 20% by mass, preferably 1.0 to 15% by mass, more preferably 0.5 to 10% by mass. If the content of the powder component is less than 0.1% by mass, a dry touch when applied to the skin cannot be obtained. Therefore, for example, it is preferable to be 0.1% by mass or more, 0.2% by mass or more, 0.3% by mass or more, 0.4% by mass or more, 0.5% by mass or more, or the like. When the content exceeds 20% by mass, the re-dispersibility tends to decrease. Therefore, for example, it is preferable to be 20% by mass or less, 18% by mass or less, 15% by mass or less, 12% by mass or less, 10% by mass or less, or the like.

The component (c) in the powder-containing cosmetic of the present invention is a fine particle powder having an average primary particle diameter of 10 to 550 nm and comprising silica or titanium dioxide as a core (hereinafter, also referred to as “fine particle powder”).

The fine particle powder in the present invention is a fine particle powder comprising silica or titanium dioxide as a core. A fine particle powder comprising silica as a core means a fine particle powder consisting of silica (silicic anhydride) or a fine particle powder comprising silica as a core and having its surface physically and/or chemically modified. Examples of the silica fine particle powder with a modified surface include fine particle powders in which the silica surface is hydrophobized with a halogenated silane, alkoxysilane, silazane, siloxane, or the like, and as a representative example there is a silylated silica in which some of the hydroxyl groups on the silica surface are substituted with a trimethylsilyl group. Furthermore, silica powders treated with various surface treating agents (described below) widely used in the field of cosmetics are included.

Similarly, a fine particle powder comprising titanium dioxide as a core also includes a fine particle powder consisting of titanium dioxide, and a fine particle powder comprising titanium dioxide as a core and having its surface physically and/or chemically modified. Examples of the surface treating agent for titanium dioxide include various surface treating agents that are widely used for the surface treatment of titanium dioxide included in cosmetics.

Specific examples of surface treatments applicable to silica and titanium dioxide include: treatments with silicones such as methyl hydrogen polysiloxane and methyl polysiloxane; fluorine treatments with perfluoroalkyl phosphate ester, perfluoro alcohol, and the like; amino acid treatments with N-acylglutamic acid, and the like; lecithin treatments; metal soap treatments; fatty acid treatments; and alkyl phosphate ester treatments.

As the fine particle powder in the powder-containing cosmetic of the present invention, one with a surface that is hydrophilic to hydrophobic, and even superhydrophobic (water repellent) can be used, but a fine particle powder having a hydrophilic to hydrophobic surface is preferably used.

The fine particle powder (component (c)) in the present invention has an average primary particle diameter of 10 to 550 nm, preferably 10 to 300 nm, more preferably 10 to 100 nm. The “average primary particle diameter” in the present invention means the diameter of primary particles measured by a method generally used in the field of cosmetics, and specifically, it is the value obtained as the arithmetic mean of the major axis and the minor axis of a particle, obtained by a transmission electron micrograph or a laser scattering/diffraction method and the like.

The average primary particle diameter of the fine particle powder needs to be 10 nm or more, and may be, for example, 15 nm or more, 20 nm or more, 25 nm or more, or 30 nm or more. Moreover, the average primary particle diameter is 550 nm or less, and may be, for example, 500 nm or less, 400 nm or less, 300 nm or less, 200 nm or less, or 100 nm or less. With a powder having an average primary particle diameter of less than 10 nm or more than 550 nm, a sufficient re-dispersibility improving effect cannot be obtained.

The shape of the fine particle powder of the present invention is not particularly limited, and may be in the form of a sphere, a plate, a rod, or the like.

The content of the fine particle powder (component (c)) included in the powder-containing cosmetic of the present invention is 0.005 to 0.2% by mass, preferably 0.01 to 0.1% by mass, more preferably 0.01 to 0.05% by mass. If the content is less than 0.005% by mass, the re-dispersibility improving effect cannot be obtained. Therefore, for example, it can be 0.008% by mass or more, or 0.01% by mass or more. On the other hand, even if the content exceeds 0.2% by mass, no further improvement of the re-dispersibility is observed.

The present invention is an invention based on finding for the first time that a fine particle powder comprising silica or titanium dioxide as a core has a function of improving the re-dispersibility of another powder component. Generally, the fact that a powder component dispersed in a liquid is stably maintained in a dispersed state is expressed as “excellent in dispersibility” or the like. However, “dispersibility” as used herein means “the dispersed state does not change (coagulation or unification does not occur) with time” and is a property that should be called “dispersion stability”. In a system excellent in “dispersion stability” (in the case of a powder of 100 nm or more), “the sediment is filled and firmly fixed, re-dispersion by stirring or the like becomes difficult, and this state is industrially called caking” (“Fundamentals of Interface and Colloid Science”, Fumio Kitahara, 2004, p. 131). That is, “re-dispersibility” in the present invention is a property that is clearly distinguished from “dispersion (stability)”, which is generally called “dispersibility”, and a system in which “dispersion (stability)” is good will have poor “re-dispersibility”.

On the other hand, fine particles having a size of 100 nm or less (sometimes referred to as “nanoparticles”) are known to exhibit a different behavior from particles having a size of several hundred nm, and to have significantly high adhesion/aggregation properties, and therefore the control of their aggregate state or dispersion is extremely difficult. It is considered that when the fine particle powder (component (c)), in which the fine particles have a size in a range including nanoparticles having such properties and having a specific material as a core, is incorporated together with another powder (component (b)) into the system of the present invention, the fine particle powder penetrates into the spaces between the precipitating (settling) other powder to prevent caking and improve “re-dispersibility”. Such an effect is an advantageous effect that is difficult to predict from the prior art.

The powder-containing cosmetic of the present invention can synergistically improve the re-dispersibility by further comprising (d) at least one, preferably two, more preferably three selected from the group consisting of a polyoxyethylene/methylpolysiloxane copolymer, calcium stearate, and sodium N,N-dimethylacrylamide-2-acrylamido-2-methylpropanesulfonate, in addition to the essential components (a) to (c).

The component (d) in the present invention, that is, a polyoxyethylene/methylpolysiloxane copolymer (component d1), calcium stearate (component d2), and sodium N,N-dimethylacrylamide-2-acrylamido-2-methylpropanesulfonate (component d3) acts as a re-dispersion promoter by combining with the fine particle powder (component (c)). When these are added, their contents are 0.1 to 1.0% by mass, preferably 0.2 to 0.6% by mass for d1 and d2, and 0.01 to

0.2% by mass, preferably 0.03 to 0.1% by mass for d3. It is preferable that the addition ratio d1:d2:d3 is 1 to 5:2 to 6:0.2 to 1, and most preferably 3:4:0.6.

The powder-containing cosmetic of the present invention can appropriately contain as needed other components that can be usually used in cosmetics, for example, moisturizing agents, oils (including silicone oil, hydrocarbon oil, ester oil, and the like), surfactants (including anionic surfactants, cationic surfactants, amphoteric surfactants, and nonionic surfactants), thickeners, ultraviolet absorbers, sequestering agents, pH adjusters, various agents, antioxidants, fragrances, water and the like, in addition to the components (a) to (d).

For example, it is preferable to add a moisturizing agent or oil that is liquid at normal temperature in order to prevent powder floating (white floating) due to the volatilization of ethanol and the like after applying the cosmetic of the present invention to the skin.

Specific examples of the moisturizing agent include: glycols such as propylene glycol, dipropylene glycol, 1,3-butylene glycol, 1,4-butylene glycol, diethylene glycol, triethylene glycol, and polyethylene glycol; glycerins such as glycerin, diglycerin, and polyglycerin; sugar alcohols such as sorbitol, mannitol, maltitol, xylitol, and erythritol; and sugars such as fructose, glucose, galactose, maltose, lactose, and trehalose. When adding a moisturizing agent to the cosmetic of the present invention, the content is usually 1 to 15% by mass, preferably 2 to 10% by mass, more preferably 3 to 8% by mass.

When adding oils to the cosmetic of the present invention, it is preferable to use silicone oil from the viewpoint of feeling of use and the like.

Examples of silicone oil include: linear polysiloxanes (for example, dimethylpolysiloxane, methylphenylpolysiloxane, diphenylpolysiloxane, and the like); cyclic polysiloxanes (for example, octamethylcyclotetrasiloxane, decamethylcyclopentasiloxane, dodecamethylcyclohexasiloxane, and the like), silicone resins, silicone rubbers, and various modified polysiloxanes that form a three-dimensional network structure (amino-modified polysiloxanes, polyether-modified polysiloxanes, alkyl-modified polysiloxanes, fluorine-modified polysiloxanes, and the like).

As other oils, examples of hydrocarbon oil include: liquid paraffin, ozokerite, squalane, pristane, paraffin, squalene, and petrolatum, and examples of ester oil include: isopropyl myristate, cetyl octanoate, octyldodecyl myristate, isopropyl palmitate, butyl stearate, hexyl laurate, myristyl myristate, decyl oleate, hexyldecyl dimethyloctanoate, cetyl lactate, myristyl lactate, lanolin acetate, isocetyl stearate, isocetyl isostearate, cholesteryl 12-hydroxystearate, ethylene glycol di-2-ethylhexanoate, dipentaerythritol fatty acid ester, N-alkylglycol monoisostearate, neopentyl glycol dicaprate, diisostearyl malate, glyceryl di-2-heptylundecanoate, trimethyloipropane tri-2-ethylhexanoate, trimethylolpropane triisostearate, pentaerythritol tetra-2-ethylhexanoate, glyceryl tri-2-ethylhexanoate, glyceryl trioctanoate, glyceryl triisopalmitate, trimethyloipropane triisostearate, cetyl 2-ethylhexanoate, 2-ethylhexyl palmitate, glyceryl trimyristate, glyceride tri-2-heptylundecanoate, castor oil fatty acid methyl ester, oleyl oleate, acetoglyceride, 2-heptylundecyl palmitate, diisobutyl adipate, N-lauroyl-L-glutamic acid-2-octyldodecyl ester, di-2-heptylundecyl adipate, ethyl laurate, di-2-ethylhexyl sebacate, 2-hexyldecyl myristate, 2-hexyldecyl palmitate, 2-hexyldecyl adipate, diisopropyl sebacate, 2-ethylhexyl succinate, and triethyl citrate.

When an oil is added, it is preferable to improve the emulsion stability by adding a surfactant. Examples of surfactants that can be added are shown below, but are not limited thereto.

Examples of anionic surfactants include: fatty acid soaps (for example, sodium laurate, sodium palmitate, and the like); higher alkyl sulfuric acid ester salts (for example, sodium lauryl sulfate, potassium lauryl sulfate, and the like); alkyl ether sulfuric acid ester salts (for example, POE triethanolamine lauryl sulfate, sodium POE lauryl sulfate, and the like); N-acyl sarcosine acids (for example, lauroyl sarcosine sodium and the like); higher fatty acid amide sulfonates (for example, N-myristoyl-N-methyltaurine sodium, coconut oil fatty acid methyltaurine sodium, lauryl methyltaurine sodium, and the like); phosphoric acid ester salts (sodium POE oleyl ether phosphate, POE stearyl ether phosphoric acid, and the like); sulfosuccinates (for example, sodium di-2-ethylhexylsulfosuccinate, sodium monolauroyl monoethanolamide polyoxyethylene sulfosuccinate, sodium lauryl polypropylene glycol sulfosuccinate, and the like); alkylbenzenesulfonates (for example, sodium linear dodecylbenzenesulfonate, triethanolamine linear dodecylbenzenesulfonate, linear dodecylbenzenesulfonic acid, and the like); higher fatty acid ester sulfuric acid ester salts (for example, sodium hydrogenated coconut oil fatty acid glycerin sulfate, and the like); N-acyl glutamates (for example, monosodium N-lauroyl glutamate, disodium N-stearoyl glutamate, monosodium N-myristoyl-L-glutamate, and the like); POE alkyl ether carboxylic acids; POE alkyl allyl ether carboxylates; α-olefin sulfonates; higher fatty acid ester sulfonates; secondary alcohol sulfuric acid ester salts; higher fatty acid alkylolamide sulfuric acid ester salts; sodium lauroyl monoethanolamide succinate; ditriethanolamine N-palmitoyl aspartate; and sodium caseinate.

Examples of cationic surfactants include: alkyltrimethylammonium salts (for example, stearyltrimethylammonium chloride, lauryltrimethylammonium chloride, and the like); alkylpyridinium salts (for example, cetylpyridinium chloride, and the like); distearyldimethylammonium dialkyldimethylammonium chloride salts; poly (N,N′-dimethyl-3,5-methylene piperidinium chloride); alkyl quaternary ammonium salts; alkyldimethylbenzylammonium salts; alkylisoquinolinium salts; dialkyl moriphonium salts; POE alkylamines; alkylamine salts; polyamine fatty acid derivatives; amyl alcohol fatty acid derivatives; benzalkonium chloride; benzethonium chloride, and the like.

Examples of amphoteric surfactants include: imidazoline amphoteric surfactants (for example, 2-undecyl-N,N,N-(hydroxyethylcarboxymethyl)-2-imidazoline sodium, 2-cocoyl-2-imidazolinium hydroxide-1-carboxyethyloxy disodium salt, and the like); and betaine surfactants (for example, 2-heptadecyl-N-carboxymethyl-N-hydroxyethylimidazolinium betaine, lauryl dimethylaminoacetate betaine, alkyl betaine, amido betaine, sulfobetaine, and the like).

Examples of lipophilic nonionic surfactants include: sorbitan fatty acid esters (for example, sorbitan monooleate, sorbitan monoisostearate, sorbitan monolaurate, sorbitan monopalmitate, sorbitan monostearate, sorbitan sesquioleate, sorbitan trioleate, diglycerol sorbitan penta-2-ethylhexylate, diglycerol sorbitan tetra-2-ethylhexylate, and the like); glycerol polyglycerol fatty acids (for example, glycerol mono-cottonseed oil fatty acid, glycerol monoerucate, glycerol sesquioleate, glycerol monostearate, glycerol α,α′-oleate pyroglutamate, glycerol monostearate malate, and the like); propylene glycol fatty acid esters (for example, propylene glycol monostearate, and the like); hydrogenated castor oil derivatives; and glycerol alkyl ethers.

Examples of nonionic surfactants include: POE sorbitan fatty acid esters (for example, POE sorbitan monooleate, POE sorbitan monostearate, POE sorbitan dioleate, POE sorbitan tetraoleate, and the like); POE sorbitol fatty acid esters (for example, POE sorbitol monolaurate, POE sorbitol monooleate, POE sorbitol pentaoleate, POE sorbitol monostearate, and the like); POE glycerin fatty acid esters (for example, POE monooleates such as POE glycerin monostearate, POE glycerin monoisostearate, and POE glycerin triisostearate); POE fatty acid esters (for example, POE distearate, POE monodioleate, ethylene glycol distearate, and the like); POE alkyl ethers (for example, POE lauryl ether, POE oleyl ether, POE stearyl ether, POE behenyl ether, POE 2-octyl dodecyl ether, POE cholestanol ether, and the like); Pluronic types (for example, Pluronic and the like); POE/POP alkyl ethers (for example, POE/POP cetyl ether, POE/POP 2-decyl tetradecyl ether, POE/POP monobutyl ether, POE/POP hydrogenated lanolin, POE/POP glycerin ether, and the like); tetra-POE/tetra-POP ethylenediamine condensates (for example, Tetronic, and the like); POE castor oil or hydrogenated castor oil derivatives (for example, POE castor oil, POE hydrogenated castor oil, POE hydrogenated castor oil monoisostearate, POE hydrogenated castor oil triisostearate, POE hydrogenated castor oil monopyroglutamic acid monoisostearic acid diester, POE hydrogenated castor oil maleic acid, and the like); POE beeswax or lanolin derivatives (for example, POE sorbitol beeswax, and the like); alkanolamides (for example, coconut oil fatty acid diethanolamide, lauric acid monoethanolamide, fatty acid isopropanolamide, and the like); POE propylene glycol fatty acid esters; POE alkylamines; POE fatty acid amides; sucrose fatty acid esters; alkylethoxydimethylamine oxides; and trioleyl phosphoric acid.

In addition, a thickener may be added to the powder-containing cosmetic of the present invention in order to improve the feeling in use (give thickness), prevent dripping after application, and the like.

The thickener used in the present invention is not particularly limited as long as it can be dissolved in a base material containing 60% by mass or more of a lower alcohol such as ethanol and exhibits a thickening effect, and can be selected from thickeners such as water-soluble polymers that are usually used in cosmetics and the like. Among these, hydroxyalkyl celluloses such as hydroxypropyl cellulose, hydroxymethyl cellulose, and hydroxyethyl cellulose are particularly preferable from the viewpoint of feeling in use.

The content of the thickener in the cosmetic of the present invention is usually 0.01 to 5.0% by mass, preferably 0.1 to 3.0% by mass. If it is less than 0.01% by mass, the effect of adding a thickener cannot be expected. Therefore, for example, it can be 0.05% by mass or more, 0.08% by mass or more, or the like. In addition, when adding a thickener, the dispersion stability of the powder improves, but the re-dispersibility tends to decrease, and when the content exceeds 5.0% by mass, the powder does not precipitate even when allowed to stand, and the multilayer type powder-containing cosmetic may not be formed. Therefore, the content of the thickener can be, for example, 4.0% by mass or less, 3.0% by mass or less, 2.0% by mass or less, or the like. It is preferable that the viscosity of the cosmetic of the present invention is adjusted to 1000 mPa·s or less, preferably 5 to 500 mPa·s, particularly preferably 10 to 300 mPa·s when a thickener is added.

Although a small amount of water may be added to the cosmetic of the present invention, the re-dispersibility tends to decrease when adding water, particularly when the component (b) is a water-soluble powder. Therefore, the cosmetic of the present invention can be an embodiment in which the content of water is, for example, 5% by mass or less, 3% by mass or less, 2% by mass or less, 1% by mass or less, or 0.5% by mass or less, or it can be an embodiment containing no water.

The powder-containing cosmetic of the present invention provides quick drying properties due to the lower alcohol included in the base material and dry touch sensation with the powder component, so that it is suitable to apply for an antiperspirant/deodorant cosmetic. In particular, it is preferable to use it as a deodorant cosmetic in which a deodorant active ingredient such as calcined alum (potassium aluminum sulfate) or an antibacterial zeolite is included as the powder component (component (b)).

The cosmetic of the present invention is provided in an aspect in which such a cosmetic is contained in a type of container correspondingly suitable to its viscosity. For example, a cosmetic prepared with a higher viscosity is suitable for being contained in jar containers and tube containers, and a cosmetic prepared with a lower viscosity can be provided in dispensers, bottles, mist containers, or roll-on containers. Both cosmetics are cosmetics of the type to be used after shaking (such as deodorant lotions), but the cosmetic of the present invention, which is excellent in re-dispersibility of the powder component can be applied evenly, without clogging the nozzles of dispensers and mist containers.

### EXAMPLES

Hereinafter, the present invention will be described more specifically with reference to Examples. The present invention is not intended to be limited by these Examples by any means. The content is indicated in % by mass with respect to the total amount, unless otherwise specified.

Using the formulae of the Examples and Comparative Examples shown in the following table, multilayer (e.g., two-layer) type powder-containing cosmetics (deodorant lotions) were produced by a conventional method, and the re-dispersibility of the powder components was evaluated in the following manner.

### <Evaluation Method>

### <Evaluation Method for the Re-Dispersibility>

A glass tube with screw cap was filled with 40 g of a sample (multilayer type powder-containing cosmetic) of each example, and allowed to stand for one month (room temperature) to precipitate (settle) the powder.

Each time the glass tube with screw cap was shaken by hand up and down five times, the re-dispersion of the precipitated (settled) powder was visually observed, and the “Re-dispersibility Index” defined below was determined.

A: number of times of shaking required for the precipitated (settled) powder to come off half of the bottom surface of the glass tube.

B: number of times of shaking required for the precipitated (settled) powder to come off the entire bottom surface of the glass tube.

Re-dispersibility Index=A×2+B

The “Re-dispersibility Index” is an index indicating the re-dispersibility suitable for the actual use of the multilayer type powder-containing cosmetic, and means that the smaller the value is, the better the re-dispersibility is.

TABLE 1

| | Comparative | Example |
|---|---|---|
| | Example 1 | 1 |
| Anhydrous Ethanol | 88.5 | 88.49 |
| Glycerin | 7 | 7 |
| Potassium Aluminum Sulfate | 4 | 4 |
| Zinc Oxide | 0.5 | 0.5 |
| Silica Dimethyl Silylate | — | 0.01 |
| (hydrophobic 0.016 μm) | | |
| Total | 100 | 100 |
| Re-dispersibility Evaluation | 60 | 30 |
| (Re-dispersibility Index) | | |

As shown in Table 1, in a system containing no thickener, in Comparative Example 1 which contains (a) a lower alcohol and (b) a powder component, but does not contain any fine particle powder (c), a Re-dispersibility Index of 60 was necessary, whereas, in Example 1 in which a fine particle powder was included, the Re-dispersibility Index was halved to 30.

TABLE 2

| | Compara- | | | |
|---|---|---|---|---|
| | tive Ex- | Exam- | Exam- | Exam- |
| | ample 2 | ple 2 | ple 3 | ple 4 |
| Anhydrous Ethanol | 87.94 | 87.93 | 87.91 | 87.91 |
| Glycerin | 7 | 7 | 7 | 7 |
| Hydroxypropyl Cellulose | 0.5 | 0.5 | 0.5 | 0.5 |
| Potassium Aluminum | 4 | 4 | 4 | 4 |
| Sulfate | | | | |
| Zinc Oxide | 0.5 | 0.5 | 0.5 | 0.5 |
| Silica | — | — | 0.03 | — |
| (spherical 0.55 μm) | | | | |
| Silica Dimethyl Silylate | — | 0.01 | — | — |
| (hydrophobic 0.016 μm) | | | | |
| Titanium dioxide | — | — | — | 0.03 |
| (0.01 μm × 0.05 μm) | | | | |
| Crosslinked Sodium N,N- | 0.06 | 0.06 | 0.06 | 0.06 |
| Dimethylacrylamide-2- | | | | |
| acrylamido-2- | | | | |
| methylpropanesulfonate | | | | |
| Copolymer | | | | |
| Total | 100 | 100 | 100 | 100 |
| Re-dispersibility Evaluation | 130 | 60 | 90 | 115 |
| (Re-dispersibility Index) | | | | |

In a system to which a thickener (hydroxypropyl cellulose) is added, the Re-dispersibility Index increases even when a re-dispersion promoter is added (Comparative Example 2), whereas the Re-dispersibility Index was significantly reduced by including a fine particle powder (c) (Examples 2 to 4).

TABLE 3

| | Comparative | | | | | | Comparative |
|---|---|---|---|---|---|---|---|
| | Example 3 | Example 5 | Example 6 | Example 7 | Example 8 | Example 9 | Example 4 |
| Anhydrous Ethanol | 87.23 | 87.22 | 87.22 | 87.22 | 87.18 | 87.13 | 87.24 |
| Glycerin | 7 | 7 | 7 | 7 | 7 | 7 | 7 |
| Hydroxypropyl Cellulose | 0.5 | 0.5 | 0.5 | 0.5 | 0.5 | 0.5 | 0.5 |
| Potassium Aluminum | 4 | 4 | 4 | 4 | 4 | 4 | 4 |
| Sulfate | | | | | | | |
| Zinc Oxide | 0.5 | 0.5 | 0.5 | 0.5 | 0.5 | 0.5 | 0.5 |
| Silica (spherical 5 μm) | 0.01 | 0.01 | 0.01 | 0.01 | 0.01 | 0.01 | — |
| Silica (hydrophilic 0.012 | — | 0.01 | — | — | — | — | — |
| μm) | | | | | | | |
| Silica Dimethyl Silylate | — | — | 0.01 | — | 0.05 | 0.1 | — |
| (hydrophobic 0.016 μm) | | | | | | | |
| Dimethicone-treated Silica | — | — | — | 0.01 | — | — | — |
| (superhydrophobic 0.012 | | | | | | | |
| μm) | | | | | | | |
| POE/Methyl Polysiloxane | 0.3 | 0.3 | 0.3 | 0.3 | 0.3 | 0.3 | 0.3 |
| Copolymer | | | | | | | |
| Calcium Stearate | 0.4 | 0.4 | 0.4 | 0.4 | 0.4 | 0.4 | 0.4 |
| Crosslinked Sodium N,N- | 0.06 | 0.06 | 0.06 | 0.06 | 0.06 | 0.06 | 0.06 |
| Dimethylacrylamide-2- | | | | | | | |
| acrylamido-2- | | | | | | | |
| methylpropanesulfonate | | | | | | | |
| Copolymer | | | | | | | |
| Total | 100 | 100 | 100 | 100 | 100 | 100 | 100 |
| Re-dispersibility | 60 | 20 | 20 | 25 | 20 | 15 | 55 |
| Evaluation | | | | | | | |
| (Re-dispersibility Index) | | | | | | | |

From the results shown in Table 3, in Comparative Example 3 which contains no fine particle powder (c), the Re-dispersibility Index was 60 even when three kinds of re-dispersion promoters ((d1), (d2) and (d3)) were added, whereas in Examples 5, 6, and 7 in which a fine particle powder comprising silica as a core (component (c)) was included, the Re-dispersibility Index was dramatically reduced to half or less. This effect was the same even when the surface properties (hydrophilic/hydrophobic/superhydrophobic) or the content of the fine particle powder were changed (Examples 5 to 9). On the other hand, by comparing Comparative Example 3 and Comparative Example 4, it can be confirmed that the blending of spherical silica with a particle diameter of 5 μm which does not correspond to the definition of the fine particle powder (c) of the present invention hardly affects the improvement of the re-dispersibility of the powder.

TABLE 4

| | Comparative | | Comparative | | Comparative | |
|---|---|---|---|---|---|---|
| | Example 5 | Example 10 | Example 6 | Example 11 | Example 7 | Example 12 |
| Anhydrous Ethanol | 87.24 | 87.23 | 87.24 | 87.23 | 87.24 | 87.23 |
| Glycerin | 7 | 7 | 7 | 7 | 7 | 7 |
| Hydroxypropyl Cellulose | 0.5 | 0.5 | 0.5 | 0.5 | 0.5 | 0.5 |
| Potassium Aluminum | 4 | 4 | 4 | 4 | 4 | 4 |
| Sulfate | | | | | | |
| Zinc Oxide | 0.5 | 0.5 | — | — | — | — |
| Polyalkyl Acrylate | — | — | 0.5 | 0.5 | — | — |
| (spherical 5 to 9 μm) | | | | | | |
| Polyalkyl Acrylate | — | — | — | — | 0.5 | 0.5 |
| (conchoidal 8 to 9 μm) | | | | | | |
| Silica Dimethyl Silylate | — | 0.01 | — | 0.01 | — | 0.01 |
| (hydrophobic 0.016 μm) | | | | | | |
| POE/Methyl Polysiloxane | 0.3 | 0.3 | 0.3 | 0.3 | 0.3 | 0.3 |
| Copolymer | | | | | | |
| Calcium Stearate | 0.4 | 0.4 | 0.4 | 0.4 | 0.4 | 0.4 |
| Crosslinked Sodium N,N- | 0.06 | 0.06 | 0.06 | 0.06 | 0.06 | 0.06 |
| Dimethylacrylamide-2- | | | | | | |
| acrylamido-2- | | | | | | |
| methylpropanesulfonate | | | | | | |
| Copolymer | | | | | | |
| Total | 100 | 100 | 100 | 100 | 100 | 100 |
| Re-dispersibility | 55 | 25 | 90 | 40 | 65 | 45 |
| Evaluation (Re- | | | | | | |
| dispersibility Index) | | | | | | |

TABLE 5

| | Example | Example | Example |
|---|---|---|---|
| | 13 | 14 | 15 |
| Anhydrous Ethanol | 85.73 | 85.73 | 85.73 |
| Glycerin | 7 | 7 | 7 |
| Hydroxypropyl Cellulose | 0.5 | 0.5 | 0.5 |
| Potassium Aluminum | 4 | 4 | 4 |
| Sulfate | | | |
| Crosslinked Silicone/ | 2 | — | — |
| Net-like Silicone Block | | | |
| Copolymer | | | |
| Talc | — | 2 | — |
| Kaolin | — | — | 2 |
| Silica Dimethyl Silylate | 0.01 | 0.01 | 0.01 |
| (hydrophobic 0.016 μm) | | | |
| POE/Methyl Polysiloxane | 0.3 | 0.3 | 0.3 |
| Copolymer | | | |
| Calcium Stearate | 0.4 | 0.4 | 0.4 |
| Crosslinked Sodium | 0.06 | 0.06 | 0.06 |
| N,N-Dimethylacrylamide- | | | |
| 2-acrylamido-2- | | | |
| methylpropanesulfonate | | | |
| Copolymer | | | |
| Total | 100 | 100 | 100 |
| Re-dispersibility Evaluation | 25 | 30 | 30 |
| (Re-dispersibility Index) | | | |

As shown in Table 4 above, an improvement of the re-dispersibility (significant decrease in the Re-dispersibility Index) was confirmed by adding a fine particle powder (c), regardless of the type (organic or inorganic) of powder constituting the powder component (b), the form of the powder, or the like. In a system further containing another powder (b), a low Re-dispersibility Index was similarly obtained (Table 5).

TABLE 6

| | Exam- | Exam- | Exam- | Exam- |
|---|---|---|---|---|
| | ple 16 | ple 17 | ple 18 | ple 19 |
| Anhydrous Ethanol | 87.93 | 87.52 | 87.62 | 87.22 |
| Glycerin | 7 | 7 | 7 | 7 |
| Hydroxypropyl Cellulose | 0.5 | 0.5 | 0.5 | 0.5 |
| Potassium Aluminum | 4 | 4 | 4 | 4 |
| Sulfate | | | | |
| Zinc Oxide | 0.5 | 0.5 | 0.5 | 0.5 |
| Silica | — | 0.01 | 0.01 | 0.01 |
| (spherical 5 μm) | | | | |
| Silica Dimethyl Silylate | 0.01 | 0.01 | 0.01 | 0.01 |
| (hydrophobic 0.016 μm) | | | | |
| POE/Methyl Polysiloxane | — | — | 0.3 | 0.3 |
| Copolymer | | | | |
| Calcium Stearate | — | 0.4 | — | 0.4 |
| Crosslinked Sodium | 0.06 | 0.06 | 0.06 | 0.06 |
| N,N-Dimethylacrylamide- | | | | |
| 2-acrylamido-2- | | | | |
| methylpropanesulfonate | | | | |
| Copolymer | | | | |
| Total | 100 | 100 | 100 | 100 |
| Re-dispersibility Evaluation | 60 | 45 | 50 | 20 |
| (Re-dispersibility Index) | | | | |

As shown in Table 6, the re-dispersibility is synergistically improved by adding at least one (that is, component (d)) selected from a polyoxyethylene/methylpolysiloxane copolymer (component d1), calcium stearate (component d2), and sodium N,N-dimethylacrylamide-2-acrylamido-2-methylpropanesulfonate (component d3), to a system in which a fine particle powder (c) is included. From the viewpoint of improving the re-dispersibility, it is clear that it is preferable to mix two rather than one of the component d1, component d2 and component d3 as the component (d), and it is further preferable to mix the three components. It was confirmed that the presence or absence of spherical silica with a particle diameter of 5 μm does not affect re-dispersibility in Comparative Examples 3 and 4 (Table 3).

TABLE 7

| | Example | Example |
|---|---|---|
| | 20 | 21 |
| Water | — | 5 |
| Anhydrous Ethanol | 81.23 | 82.23 |
| Glycerin | 4 | 7 |
| Hydroxypropyl Cellulose | 0.5 | 0.5 |
| Methylphenyl Polysiloxane | 3 | — |
| Decamethylcyclopentasiloxane | 3 | — |
| Methyl Polysiloxane | 3 | — |
| Potassium Aluminum Sulfate | 4 | 4 |
| Zinc Oxide | 0.5 | 0.5 |
| Silica (spherical 5 μm) | — | — |
| Silica Dimethyl Silylate | 0.01 | 0.01 |
| (hydrophobic 0.016 μm) | | |
| POE/Methyl Polysiloxane | 0.3 | 0.3 |
| Copolymer | | |
| Calcium Stearate | 0.4 | 0.4 |
| Crosslinked Sodium N,N- | 0.06 | 0.06 |
| Dimethylacrylamide-2- | | |
| acrylamido-2- | | |
| methylpropanesulfonate | | |
| Copolymer | | |
| Total | 100 | 100 |
| Re-dispersibility Evaluation | 30 | 35 |
| (Re-dispersibility Index) | | |

Moreover, the same re-dispersibility was exhibited even when oils were added to the formula of Example 10 (Re-dispersibility Index=25) (Example 20). On the other hand, when water (5% by mass) was added to the formula of Example 10, the re-dispersibility was slightly reduced (Example 21), but compared to Comparative Example 4 containing no fine particle powder (c) (Re-dispersibility Index=55), the re-dispersibility was dramatically improved, and such a level is deemed not practically problematic.

1. A multilayer powder-containing cosmetic, comprising:
(a) 60 to 95% by mass of a lower alcohol;
(b) a powder component; and
(c) a fine particle powder having an average primary particle diameter of 10 to 550 nm;
wherein said fine particle powder (c) further comprising:
at least a powder selected from a group consisting of silica and titanium dioxide that forms a core of the fine particle powder; and
wherein, said powder component (b) is different from said fine particle powder component (c).

2. The cosmetic, according to claim 1, wherein:
said fine particle powder (c) comprises said silica in said core.

3. The cosmetic, according to claim 1, further comprising:
(d) at least one component selected from a group consisting of a polyoxyethylene/methylpolysiloxane copolymer, calcium stearate and sodium N,N-dimethylacrylamide-2-acrylamido-2-methylpropanesulfonate.

4. The cosmetic, preparation according to claim 1, wherein:
said powder component (b) comprises:
at least one powder selected from a group consisting of potassium aluminum sulfate, zinc oxide, an extender pigment, silica having an average primary particle diameter larger than 0.55 μm, titanium dioxide having an average primary particle diameter larger than 0.55 μm, a polyalkyl acrylate powder, a crosslinked silicone powder and a nylon powder.

5. The cosmetic, according to claim 1, wherein:
said cosmetic is a deodorant that comprises potassium aluminum sulfate as the powder component (b).

6. The cosmetic, according to claim 1, wherein:
said cosmetic is filled in a roll-on container.

7. The cosmetic, according to claim 1, wherein:
a viscosity of said cosmetic is not more than 1000 mPa·s.
